(19) United States
(12) Patent Application Publication
(10) Pub. No.: US 2007/0203517 A1
(43) Pub. Date: Aug. 30, 2007

(54) Transgastric surgical devices and procedures

(76) Inventors: Michael S. Williams, Santa Rosa, CA (US); Richard S. Stack, Chapel Hill, NC (US); Geoffrey A. Orth, Sebastopol, CA (US); Jeff Smith, Petaluma, CA (US); Richard A. Glenn, Chapel Hill, NC (US); William L. Athas, Chapel Hill, NC (US); Aurora Pryor, Durham, NC (US); Daniel W. Fifer, Windsor, CA (US)

Correspondence Address: STALLMAN & POLLOCK LLP, 353 SACRAMENTO STREET, SUITE 2200, SAN FRANCISCO, CA, 94111, US

(21) Appl. No.: 11/528,009

(22) Filed: Sep. 27, 2006

Related U.S. Application Data
(60) Provisional application No. 60/720,943, filed on Sep. 27, 2005.
(60) Provisional application No. 60/794,563, filed on Apr. 24, 2006.
(60) Provisional application No. 60/826,535, filed on Sep. 21, 2006.

(51) Int. Cl. A61M 29/00

(52) U.S. Cl. 606/191

(57) ABSTRACT

Embodiments of surgical access cannulas and access systems for use in gaining access to a body cavity of a patient via a natural orifice are disclosed. A distal end of an access cannula is advanced through a natural orifice into a hollow organ. Instruments passed through the cannula are used to form an incision in the wall of the hollow organ. The access cannula is anchored in the incision with its distal opening giving access to a body cavity outside the hollow organ. Surgical instruments are passed through the access cannula and used to perform procedures in the body cavity.

### RELATED APPLICATIONS

This application claims the benefit of U.S. Provisional Application No. 60/720,943, filed Sep. 27, 2005, U.S. Provisional Application No. 60/794,563, filed Apr. 24, 2006, U.S. Provisional Application No. 60/826,535, filed Sep. 21, 2006.

### FIELD OF THE INVENTION

The present invention relates to the field of access devices and procedures for use in performing surgery in the peritoneal cavity.

### BACKGROUND OF THE INVENTION

Surgery in the abdominal cavity is typically performed using open surgical techniques or laparoscopic procedures. Each of these procedures requires incisions through the skin and underlying muscle and peritoneal tissue, and thus results in the potential for post-surgical scarring and/or hernias.

Systems and techniques in which access to the abdominal cavity is gained through a natural orifice are advantageous in that incisions through the skin and underlying muscle and peritoneal tissue may be avoided. Use of such systems can provide access to the peritoneal cavity using an access device inserted into the esophagus, stomach or intestine (via, for example, the mouth or rectum). Instruments are then advanced through the access device into the peritoneal cavity via an incision in the wall of the esophagus, stomach or intestine. Other forms of natural orifice access, such as vaginal access, may similarly be used.

### BRIEF DESCRIPTION OF THE DRAWINGS

FIG. 1 is a side elevation view showing one embodiment of a surgical access cannula.

FIG. 2 is a cross-sectional top view taken along the plane designated 2-2 in FIG. 1.

FIG. 3 is a perspective view of the instrument/scope port of the cannula of FIG. 1.

FIG. 4 is a perspective view of the distal portion of the cannula of FIG. 1, including the valve and anchors.

FIG. 5A is a side elevation view of the distal portion of the cannula of FIG. 1.

FIG. 5B is a view similar to the view of FIG. 5A showing detachable anchoring elements on the distal end of the cannula.

FIG. 6 is a perspective view showing alternate anchoress suitable for use on the cannula of FIG. 1.

FIG. 7 is a perspective view of the seals of FIG. 6 mounted on the cannula.

FIG. 8A is a cross-sectional side view of the distal end of an access cannula showing an alternative anchor design. FIG. 8B is a side elevation view of the anchor of FIG. 8A in the expanded position.

FIGS. 9A and 9B are cross-sectional side views of the distal end of an access cannula showing another alternative anchor design.

FIG. 10A is a side elevation view of the distal end of an access cannula showing yet another anchor design. FIG. 10B is a cross-sectional side view of the distal end shown in FIG. 10A, showing the anchor in the expanded position.

FIG. 11A is a cross-sectional side view of the distal end of an access cannula showing another anchor design. FIG. 11B is a side elevation view of the anchor of FIG. 11A in the expanded position.

FIG. 12A is a side elevation view of a distal end of a cannula having a tapered obturator tip and a threaded anchor. FIG. 12B is a similar view showing a threaded anchor only on the cannula shaft.

FIGS. 13A through 13H are a sequence of drawings illustrating one method of placing the access cannula of FIG. 1.

FIGS. 14A through 14C are a sequence of schematic drawings illustrating an alternative placement method for the cannula of FIG. 1 and its use to perform surgery in the abdominal cavity.

FIG. 15 is an exploded side elevation view of an access system in which the access cannula and septum are shown in cross-section.

FIG. 16 is a partial cross-sectional side view showing the cannula and obturator tip of FIG. 15 assembled for use.

FIGS. 17A through 17K are a sequence of side views showing use of the access system of FIG. 16. In FIGS. 17A, 17B, 17D, 17F, 17H and 17J the cannula is shown in cross-section. In FIGS. 17C, 17E, 17G, 17K, 17K the cannula is shown in cross-section and the stomach wall is not visible.

FIGS. 18 and 19 are views similar to FIG. 17F showing alternative balloon dilator configurations.

FIGS. 20A through 20B are a sequence of perspective drawings illustrating use of an alternative access system.

FIG. 21A is a cross-sectional side view showing an alternative embodiment of an access system. FIGS. 21B, 22A and 22B illustrate use of the system of FIG. 21A.

FIG. 23 is a cross-sectional side view of an alternative access system.

FIG. 24 is a perspective view of yet another access system.

FIG. 25A is a front plan view of a first embodiment of a closure device.

FIG. 25B is a side elevation view of the closure device of FIG. 25A.

FIG. 25C is a perspective view of the closure device of FIG. 25A.

FIG. 25D is a top view of the closure device of FIG. 25A.

FIGS. 25E and 25F are a top view and a side elevation view of the closure device of FIG. 25A after each wing has been folded in preparation for insertion of the closure device into a delivery tube.

FIG. 25G is similar to FIG. 25F and shows the closure device following a second folding step.

FIG. 26 is a perspective view showing the closure device of FIG. 25A in a folded configuration and positioned next to a deployment system for use is placing the closure device in an abdominal wall incision.

FIGS. 27 through 33 are a sequence of perspective drawings illustrating deployment of the closure device of FIG. 25A using the FIG. 26 system. FIGS. 34 and 35 are side elevation views of an alternative embodiment of a surgical access cannula, in which use of the cannula is illustrated.

FIG. 36 is a schematic drawing illustrating use of the cannula of FIG. 1 in performing surgery on a portion of a bowel.

FIG. 37A is a side elevation view illustrating components of a system used to facilitate visual inspection of an intestine. FIG. 37B illustrates the arrangement of the components of the FIG. 37A system during use.

FIGS. 38-42 are a sequence of schematic drawings illustrating use of the intralumenal inspection system of FIG. 12A in the intestine of a human patient.

### DETAILED DESCRIPTION OF THE DRAWINGS

Generally speaking, the present application describes embodiments of surgical access cannulas and access systems for use in gaining access to a body cavity of a patient via a natural orifice. The cannula is configured such that its distal end may be advanced through a natural orifice (e.g. mouth, rectum, vaginal opening) into a hollow organ (esophagus, stomach, intestine, vagina or uterus). Once the cannula is positioned in the hollow organ, instruments passed through the cannula are used to form an incision in the wall of the hollow organ. Elements of the cannula create sealed access through the incision, permitting preferably sterile passage of instruments into the peritoneal cavity. The application also describes a system allowing intralumenal inspection of a patient's intestine using transoral access. This system may be used in procedures utilizing the disclosed access cannula, as well as in separate procedures.

The disclosed devices, systems and methods are described with respect to transgastric access to the peritoneal cavity. This is by way of example only, as the disclosed embodiments are equally suitable for other natural orifice procedures.

Procedures within the body that can be performed using natural orifice access include but are not limited appendectomy, cholecystectomy, hysterectomy, oopherectomy, and treatment of the intestine and prostate.

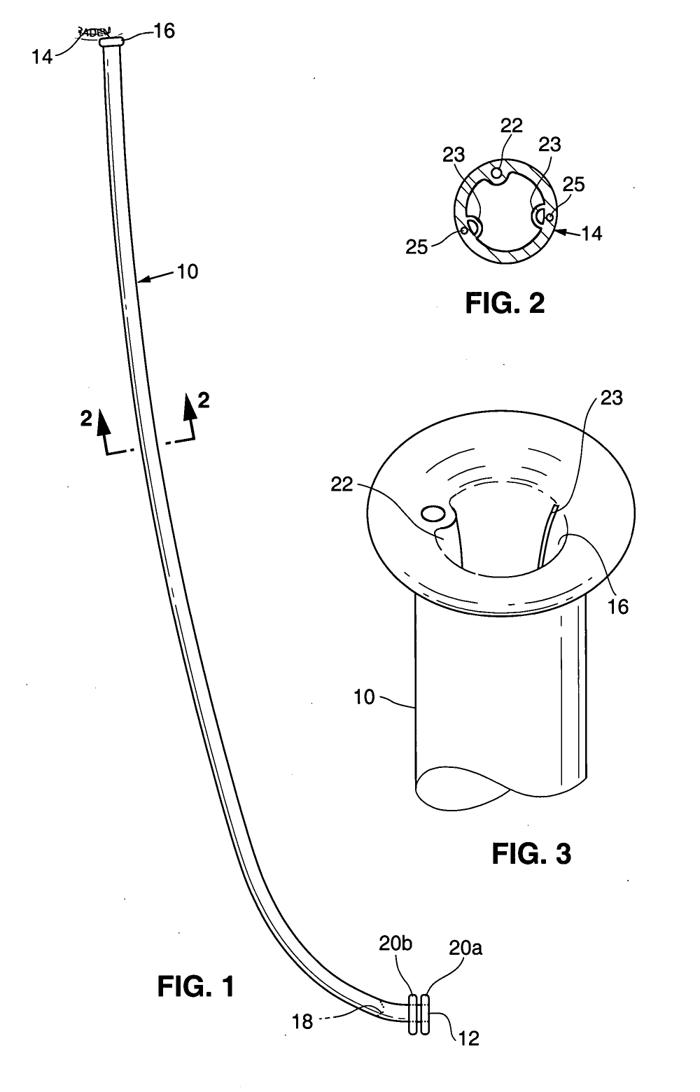

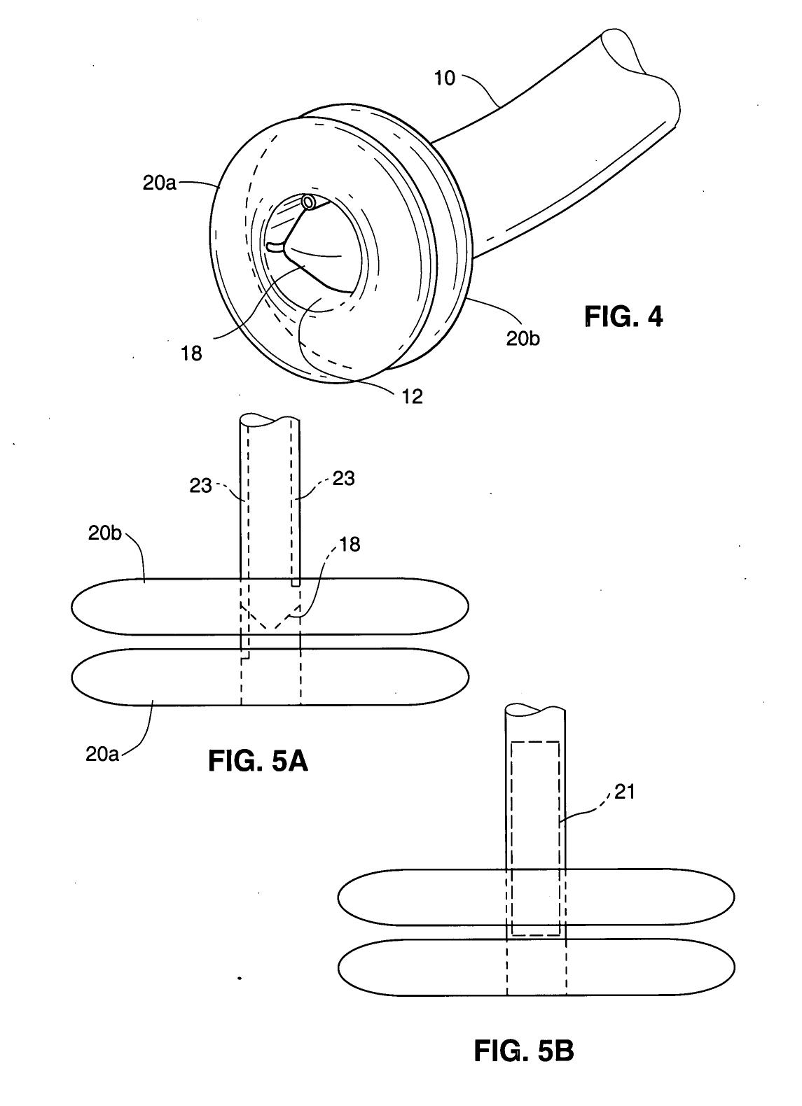

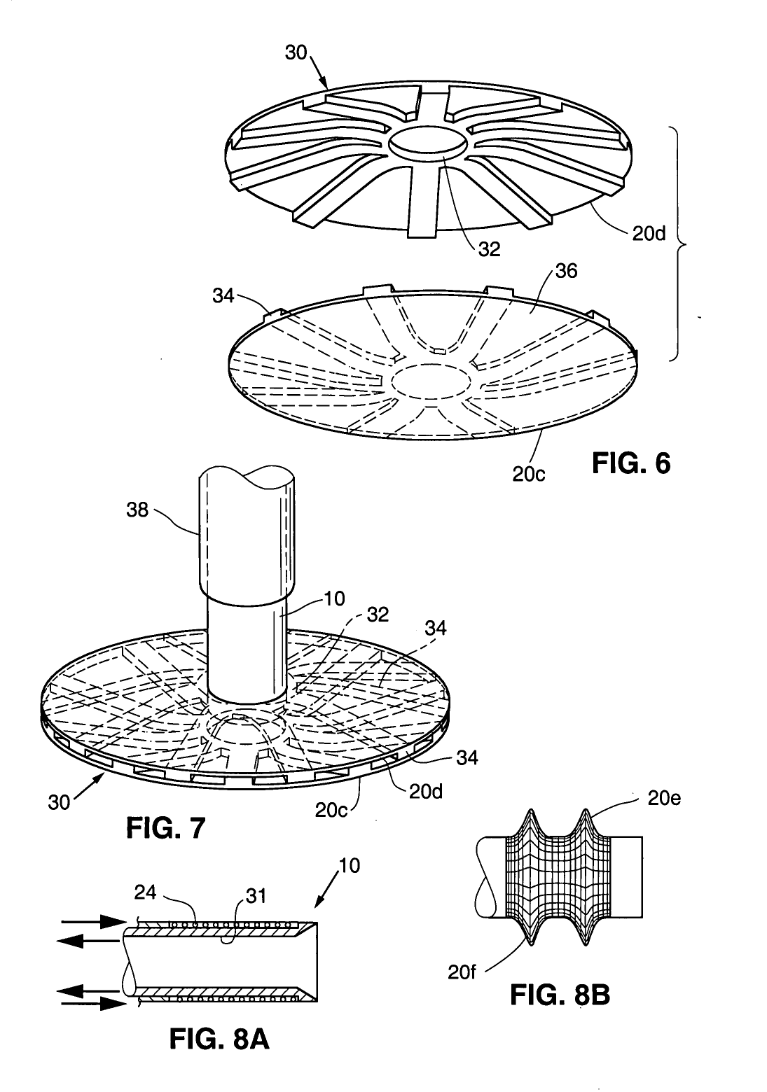

Referring to FIG. 1, one embodiment of a transgastric access device includes an elongate cannula 10 having at least one working lumen 14 extending the length of the cannula to a distal port 12. An instrument port 16 is formed at the proximal end of the lumen, and a valve 18 is positioned to seal the distal portion of the lumen. A pair of sealing elements 20a, 20b are positioned on the exterior of the cannula 10, near the distal port 12. As discussed in connection with FIGS. 4 and 6, the sealing elements may comprise inflatable balloons or other elements capable of anchoring the cannula within an incision formed in a stomach wall and preferably forming a seal between the cannula and the incision.

In one embodiment, the working lumen 14 may be a single lumen of a size appropriate for receiving instruments needed for the procedure, as shown in FIG. 2. Alternate embodiments may include two or more lumens.

FIG. 3 illustrates the proximal portion of the system, which during use is positioned with the instrument port 16 in the mouth or outside of the mouth with the cannula 10 extending down the esophagus to the stomach. A light source lumen 22 extends the length of the cannula. The light source lumen includes fiber optic elements coupled to a fiber optic lighting system or other suitable lighting source (not shown) so as to permit illumination of the procedure to be carried out at the distal end of the cannula 10. If the anchoring elements 20a, 20b (FIG. 1) are inflatable, inflation ports 23 (FIGS. 2 and 3) provide a conduit for delivery of inflation fluid or gas into the balloons using an inflation device such as a syringe (not shown) or other suitable inflation system. If a deflectable/steerable cannula is desired, pullwires 25 (FIG. 2) extend through corresponding pullwire lumens in the cannula 10 and are anchored within the cannula's distal region.

Referring to FIG. 4, valve 18 may be positioned within the cannula 10, near the distal port 12 as shown, or in a more proximal portion of the cannula 10. The valve 18 may take the form of a duck bill valve as shown, or any other type of valve suitable for sealing the distal portion of the lumen 14 in the absence of an instrument through the lumen. The valve 18 can thus prevent movement of fluids and/or gases into the lumen during passage of the distal port 12 through the stomach and into the peritoneal cavity. The valve may additionally be configured such that it will seal against instruments passed through the valve 18, thus preventing movement of fluids and gases around instruments extending through the valve 18 and preventing loss of insufflation pressure from the peritoneal cavity is insufflation is used. In alternative embodiments, a separate valve or seal may be mounted within the lumen 14 for use in forming a seal around the periphery of instruments passed through the lumen 14. Valves and seals useful for these purposes include those of the type used in trocars commonly used in laparoscopic surgical procedures.

Anchoring elements 20a, 20b may be inflatable annular cuffs as shown in FIG. 5. Each such anchoring element is fluidly coupled to a corresponding one of the inflation ports 23 (FIG. 2), so that the anchoring elements 20a, 20b may be separately inflated. Anchoring elements 20a, 20b are formed of a durable polymeric material, and are spaced from one another along the length of the cannula 10 so as to allow them to be positioned on opposite sides of a portion of stomach wall.

In an alternative embodiment, the anchoring elements 20a, 20b are detachable from the cannula 10 so that they might be left in place against the stomach wall to continue to seal the incision formed in the stomach wall. For example, as shown in FIG. 5B, the distal end of the cannula may be sealed using a closure pin 21 or other device positioned within the lumen of the cannula, and a distal portion of the cannula 10 (where the anchoring elements are positioned) may be detachable from the remainder of the cannula 10. According to this alternative embodiment, the portions of the cannula that are to remain within the body may be formed of bioerodible material that will passively degrade at some point after the incision in the stomach wall has healed or actively degrade once exposed to heat, light, electrical energy or certain chemical agents. Detachable anchoring elements might also include have drug delivery capability via a coating matrix impregnated with one or more pharmaceutical agents, including therapeutic agents and/or agents selected to promote healing of the incision or ingrowth of tissue onto the anchoring elements.

FIGS. 6 and 7 illustrate an access cannula using alternative anchoring elements 20c, 20d, each of which includes a frame member 30 that may include a central ring 32 mounted to the cannula 10 (FIG. 7), and radial members 34 extending from the ring 32. The frame members 30 may be formed of a shape memory material such as nitinol or shape memory polymer, or other material that allow the anchoring elements 20c, 20d to be compressed into to a delivery sheath 38 (FIG. 7) but that will allow the anchoring elements 20c, 20d to spring to their expanded position once released from the delivery sheath 38. A polymeric disk 36 is mounted to the frame member 30.

Other anchoring systems are illustrated in FIGS. 8A through 12B. The illustrated systems may provide only distal anchoring (i.e. an anchor against the exterior of the stomach wall) to prevent the cannula 10 from pulling out of the incision in the stomach wall, or they may provide both proximal and distal anchoring similar to that provided by balloons 20a, 20b of FIG. 1 to also prevent inadvertent advancement of the cannula further into the peritoneum. Preferred anchoring systems will also seal the periphery of the incision to prevent material from within the stomach from contaminating the sterile peritoneal cavity, however as an alternative the portion of the cannula that seats within the incision may have a compliant exterior surface that itself forms a seal with the incision.

Referring to FIG. 8A, the cannula 10 may have a distal portion having a tubular length of braid 29 overlaying a shaft 31. Braid 29 is shaped such that at least a portion of it will expand outwardly to form anchors 20e, 20f as shown in FIG. 8B when shaft 31 is withdrawn relative to the braid 29.

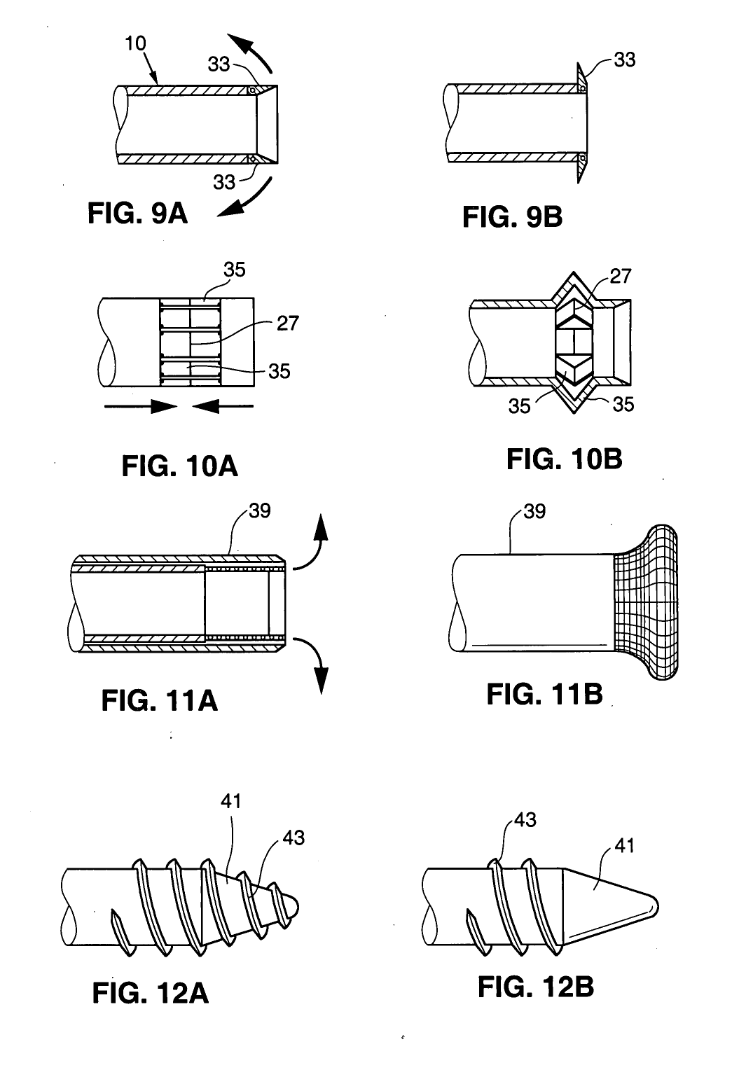

In the FIG. 9A embodiment, the distal portion of the cannula 10 includes a hinged annular collar 33 that self-expands or is actively pivoted to the radially extended position shown in FIG. 9B. The FIG. 10A embodiment includes longitudinal strips 35 cut into the distal portion of the cannula 10. Strips 25 bow outwardly as shown in FIG. 10B when the distal end of the cannula is longitudinally compressed. Compressive forces can be applied in a number of ways, such as by applying tension to pullwires connected to the distal end of the cannula while pushing against the proximal end of the cannula, or by pushing against the cannula while supporting the distal end of the cannula using an instrument passed through the lumen of the cannula. Circumferential folds lines or weakened regions 27 may be formed in the strips such that the strips will crease at selected locations.

In another alternative anchoring system shown in FIGS. 11A and 11B, the distal end of the access cannula 10 may have a braided distal end that can be made to self-expand (e.g. upon withdrawal of a sheath 39) to a flared “trumpet” configuration (FIG. 11B) outside the stomach wall. The cannula may optionally include a corresponding lip (which may be preformed or self expandable) spaced from the distal end and positionable inside the stomach wall, such that the wall is retained between the flare and the lip.

In another embodiment shown in FIG. 12A, cannula 10 includes a tapered tip 41 having helical ribs 43 or threads on the cannula shaft and the tip 41, or only on the shaft as in the FIG. 12B embodiment. These embodiments allow simultaneous advancement of the cannula through an incision, dilation of the incision, and anchoring of the cannula within the incision. Tip 41 may be retractable to open the cannula, following anchoring, for passage of instruments. Other retractable tips are described below.

The access cannula 10 may be a flexible tube formed of polymeric material (e.g. polyurethane). The cannula 10 may be highly compliant for introduction into the body, allowing the cannula to be partially or fully collapsed for delivery into the stomach. The cannula's properties can be tailored for optimal radial strength, compliance and bending radius. A compliant cannula may be supported during or after passage into the stomach by a secondary structure such as the access system (e.g. obturators of the type discussed below) or by other instruments inserted into the cannula.

Materials useful for the cannula include ePTFE, woven materials such as polyester, polyurethane, composite materials (e.g. lycra with polyester) as well as others. A lubricious material such as ePTFE will provide a lubricious surface for ease of delivery through the esophagus and passage of instruments through the cannula. In some embodiments, all or a portion of the cannula may include microporous regions having a pore size that allows therapeutic or antiseptic solutions to be administered to the surrounding area while preventing flow of contaminants into the cannula. For example, a solution may be directed under pressure through the cannula, causing the solution to pass through the pores in the walls of the cannula. Alternative cannula embodiments may be reinforced using various materials. Reinforcements may be continuous, variable, or site specific along the length of the cannula.

The cannula may be a polymeric material reinforced with an internal, external, or embedded spiral wrapped coil (e.g. flat or round wire of stainless steel, nitinol or suitable alternatives, monofilament of polyester, nylon etc, or other material). The spiral wrap reinforcement provides radial strength allowing for an improved bend radius. A tightly wound (e.g. closed) coil improves the axial stiffness of the cannula, which may improve column strength for advancing the cannula, actuating anchoring systems, or improving advancement of instruments through the cannula.

In other embodiments, an internal, external or embedded braided structure may be on or in the walls of the cannula to improve radial strength, column strength, and torsional stiffness. Braid structures may be additionally be used to make the cannula compressible to a reduced diameter (such as through the application of longitudinal tension on the braid) or expandable (through longitudinally compression of the braid. Expandable braid features may be used to anchor the cannula within an incision as discussed above. Exposed braid on the exterior of the cannula may provide additional traction for anchoring.

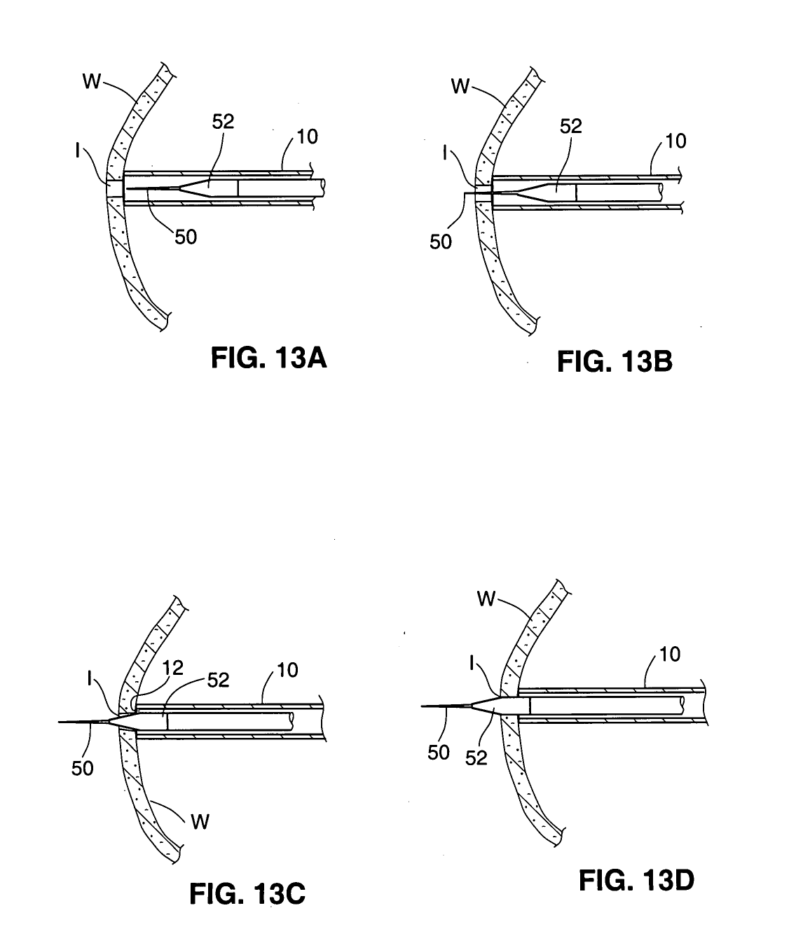

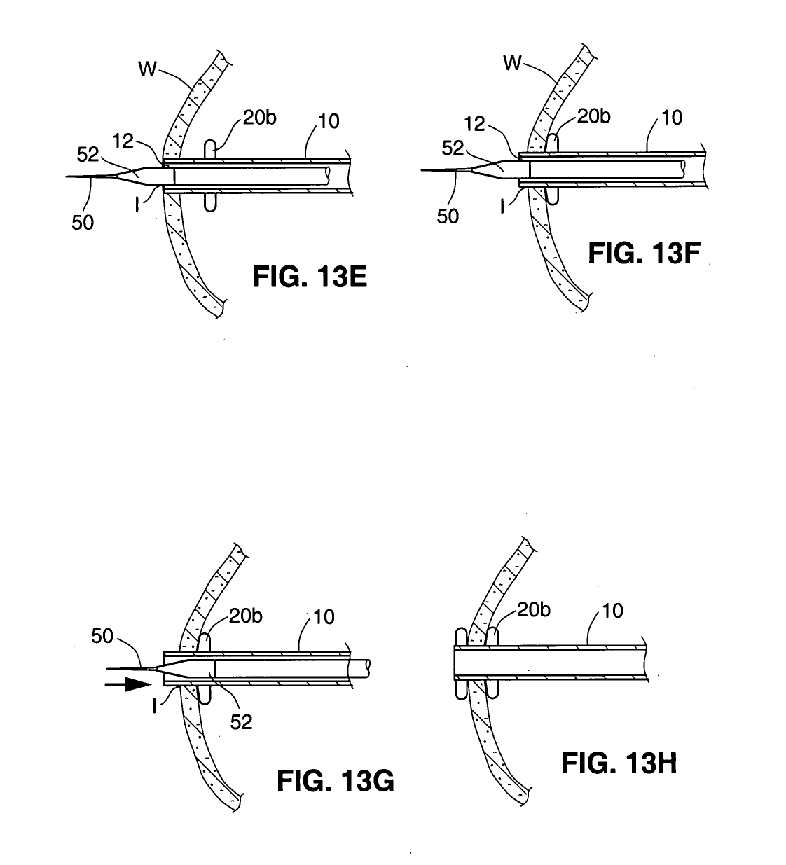

A method for using the access cannula 10 includes passing the distal end 12 of access cannula 10 into the mouth of a patient, through the esophagus E, and into the stomach S (or, in alternative embodiments, into the intestine via the rectum, or through the vagina for access through the vaginal ceiling or the uterus). Referring to FIGS. 13A and 13B, with the cannula 10 preferably in contact with the wall W to be penetrated, an incision I or perforation is formed in the wall W using an instrument such as a needle 50 passed through the cannula 10.

Once an incision is made using the needle, it may be necessary to pass a dilator through the incision to expand the incision I. In the embodiment shown, needle 50 extends from the distal end of a dilator 52, which is pushed through the incision I to expand the incision as shown in FIGS. 13C and 13D. In an alternative embodiment discussed below, the needle may be protected within the lumen of the dilator as it is advanced through the access cannula, and then advanced from the dilator to form the incision I. Small knife edges (not shown) may extend from the surface of the dilator to allow the incision to be expanded by cutting, thus minimizing trauma to the wall. In other alternatives, the dilator may have an expandable portion incorporating inflatable balloons, expandable shape-memory braid sections, or other expandable features that may be positioned within the incision I and then expanded to increase the size of the incision. The dilator may further incorporate an endoscope to give the practitioner visual feedback as s/he forms the incision and anchors the access cannula.

The distal end 12 of the cannula 10 is advanced into the incision I, and proximal anchoring element 20b on the cannula is inflated as shown in FIG. 13E. Next, the distal end 12 of the cannula 10 is passed fully through the incision I as shown in FIG. 13F, such that distal anchoring element 20a (which at this point is uninflated) on the cannula is positioned outside of the stomach and proximal most anchoring element 20b on the cannula remains inside the stomach, preferably in contact with wall W. The dilator 52 and needle 50 are withdrawn from the body as illustrated in FIG. 13G. Inflation fluid is delivered to inflate the distal anchoring element 20a as shown in FIG. 13H, causing the wall W to be engaged between the anchoring elements 20a, 20b, and further causing the anchoring elements 20a, 20b to seal the incision I against passage of fluids and/or gases. Once anchored in place, the access cannula provides sterile access to the peritoneal cavity. Instruments to be used to perform a procedure within the peritoneal cavity are thus passed into the proximal end of the access cannula which remains outside the body, and advanced through the cannula into the peritoneal cavity.

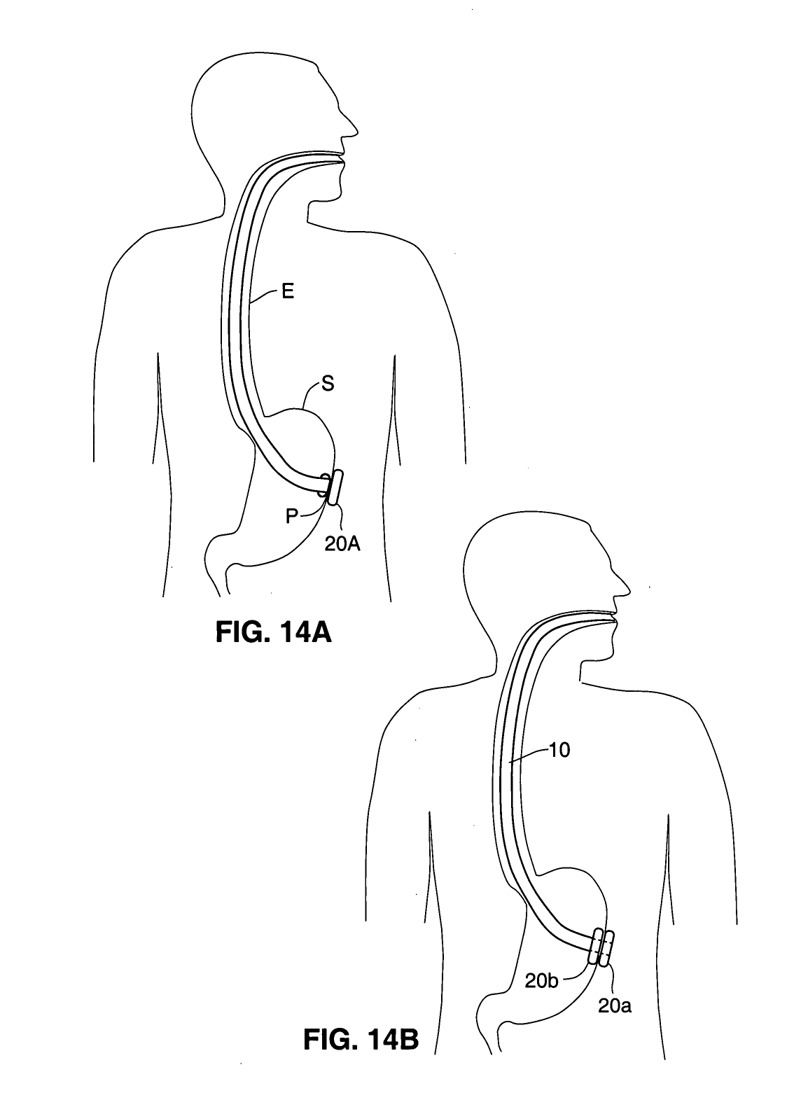

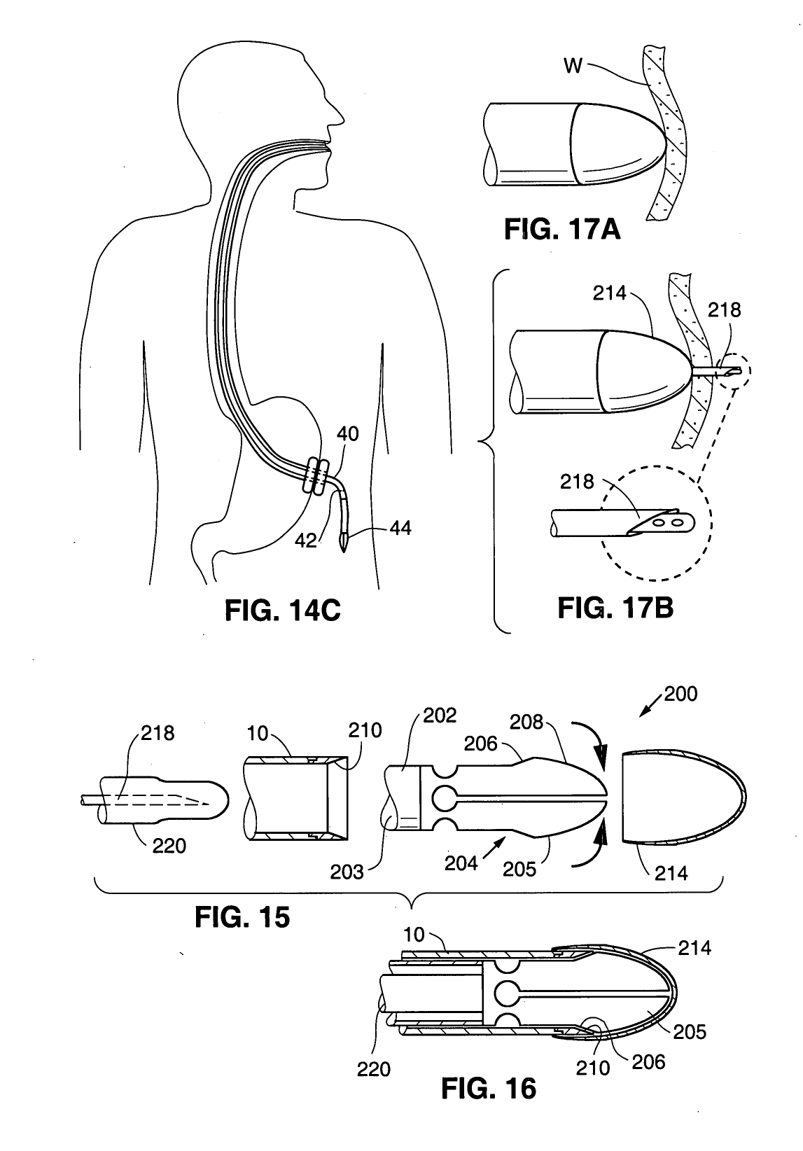

In an alternative method for placing the access cannula of FIG. 1, the distal portion of the cannula 10 is passed through the incision I, such that the distal most anchoring element 20a is positioned outside of the stomach and the proximal most anchoring element 20b remains inside the stomach. Inflation fluid is delivered to inflate the distal anchoring element 20a as shown in FIG. 14A. If the embodiment of FIG. 7 is instead used, the cannula 10 is introduced into the stomach while disposed inside the sheath 38, with the anchoring elements 20c, 20d in a compressed orientation inside the sheath 38. The sheath 38 (with the cannula 10 inside it) is passed through the perforation P. The cannula 10 is advanced slightly in a distal direction to release the distal most anchoring element 20c from the distal end of the sheath, causing the anchoring element 20c to expand.

Referring to FIG. 14B, once the distal anchoring element 20a has been inflated, traction is applied to the cannula 10 to draw the distal anchoring element 20a into firm contact with the stomach wall. Next, inflation fluid is delivered to inflate the proximal anchoring element 20b, causing the stomach wall to be engaged between the anchoring elements 20a, 20b, and further causing the anchoring elements 20a, 20b to seal the perforation P against passage of fluids and/or gases. If the FIG. 7 embodiment is used, deployment of the proximal anchoring element 20d of the FIG. 7 embodiment is achieved by withdrawing the sheath 38 proximally to release the anchoring element 20d, thus causing the stomach wall to be engaged between the anchoring elements 20c, 20d.

Finally, referring to FIG. 10, a procedural cannula 40 is passed through the cannula 10. Procedural cannula 40 preferably includes a valve 42 sealing its distal end against passage of fluids. Valve 42 may be a duckbill type valve as described above, and/or one which will seal around instruments passed through it, each of which is commonly found in laparoscopic trocars. Instruments 44 needed to perform the desired procedure within the peritoneal cavity (e.g. forceps, electrosurgical tools, snares, cutters, endoscopes, staplers etc.) are passed through the access cannula 40 and used to carry out the procedure. Once the procedure has been completed, the procedural cannula 40 and instruments are removed, anchoring elements 20a, 20b are deflated (or, in the case of anchoring elements 20c, 20d of FIG. 7, withdrawn into sheath 38), and the cannula 10 is removed from the body.

Ease of passage of the cannula 10 through the esophagus (or intestine) may be enhanced through the use of an access system employing an obturator. One access system comprising an access cannula 10 and obturator 200 is shown in FIG. 15. Obturator 200 includes an elongate tubular shaft 202 that extends through the cannula 10 out of the patient, and a tip 204 on the distal end of the obturator. A passage or lumen 203 extends through the shaft 202 and the tip 204. Tip 204 preferably includes a proximal portion 206 that flares outwardly from the shaft 202, and a tapered distal portion 208. The shaft 202 is preferably formed of braided tubing or other materials that give sufficient column strength, a desired bend radius, torsional stiffness for movement through the target region of the body (e.g. esophagus, intestine). Suitable examples include those listed with respect to reinforced cannula designs.

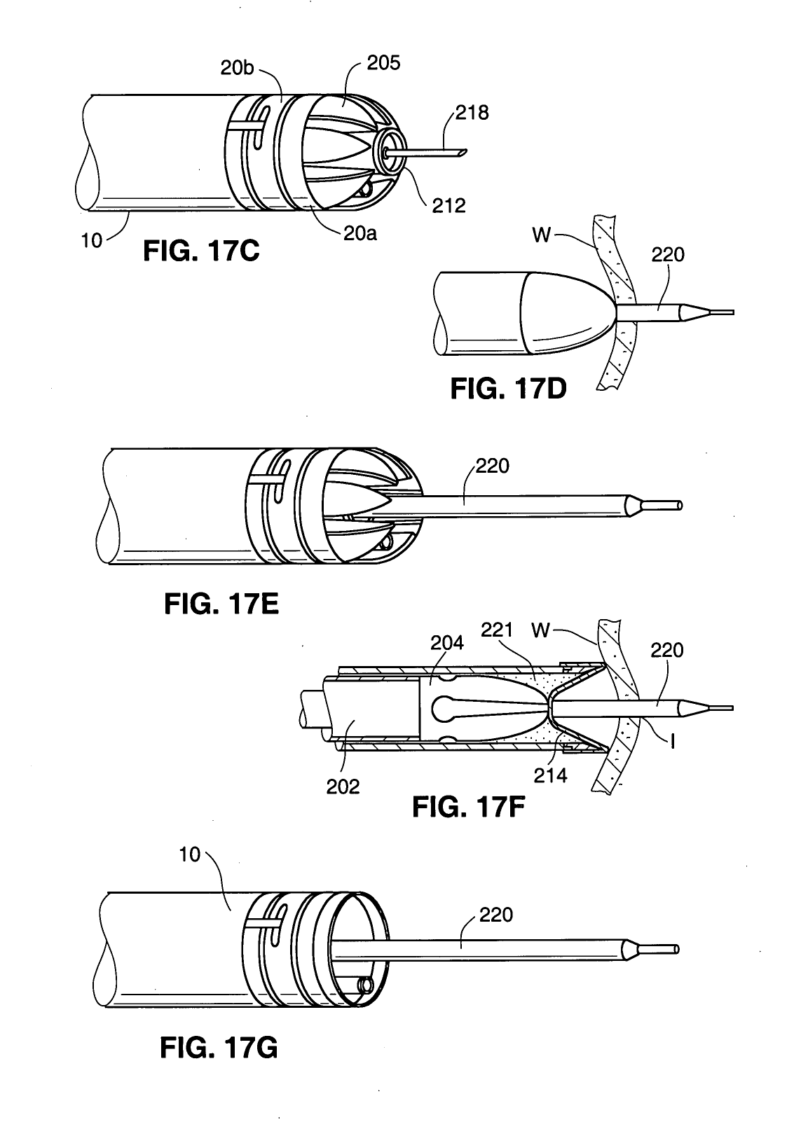

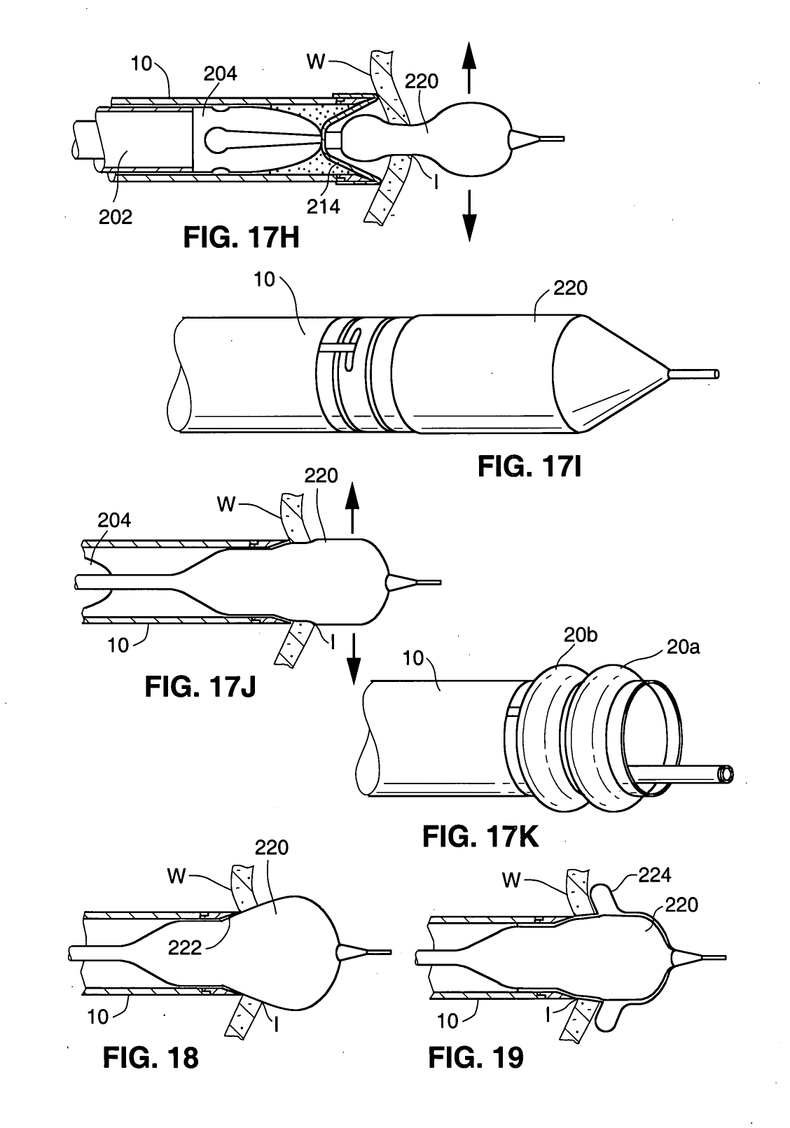

Tip 204 is divided into a number of circumferentially spaced spring elements 205. FIG. 15 illustrates that the cannula 10 may include a beveled distal edge 210 on its interior lumen, such that when the obturator 200 is disposed within the cannula 10 as shown in FIG. 16, the flared proximal portion 206 of the tip is adjacent to the beveled edge 210 of the cannula 10. A locking element 212 (FIG. 17B) positioned within the lumen 203 of the obturator 200 urges the spring elements 205 outwardly into contact with the beveled edge 210 so as to prevent the obturator 200 from moving in a proximal direction within the cannula. The locking element 212 is shown as a tube, but it may be any other feature that will lock the obturator in its distal position.

A dilation balloon catheter 220 is advanceable through the cannula 10 and obturator 200. A needle 218 is extendable through a lumen in the balloon catheter 220, or it may be an extendable and retractable component of the balloon catheter 220.

The obturator system of FIG. 16 allows the access cannula to be aseptically positioned within a stomach wall incision. As shown, a transparent septum 214 covers the obturator and is sealed around the circumference of the cannula. The septum 214 seals the distal ends of the obturator and cannula so as to maintain a sterile environment within the cannula allowing clean passage of instruments into the peritoneal space. The transparent material of the septum allows visualization of structures outside the distal end of the obturator 200 and cannula 210 using endoscope 216. Septum 214 is preferably coupled to the obturator tip 202.

According to one method of placing the cannula 10 using the access system of FIG. 16, the system is advanced through the esophagus and into contact or close proximity with the stomach wall W under visualization using endoscope 16 (FIG. 17A). Needle 218 is advanced through the cannula and out the distal end of the obturator, perforating both the septum 214 (see FIGS. 15 and 16) and the stomach wall W. (FIGS. 17B and 17C). If insufflation is needed for visualization within the peritoneal cavity, the cavity may be insufflated using gas directed through the needle 218.

Balloon dilator 220 is advanced through the incision I (FIG. 17D) and the locking element 212 is retracted (FIG. 17E). A stream 221 of sterile saline or other substance (e.g. antiseptic) may be directed through the cannula 10 to the stomach wall or incision during any part of the procedure.

The obturator tip 204 is retracted as shown in FIGS. 17F and 17G by sliding the shaft 202 of the obturator in a proximal direction. Retraction of the obturator tip 204 also retracts the septum 214 as shown. The balloon 220 is expanded to dilate the incision I. FIGS. 17 H-17I. The beveled edge of the cannula and expansion of the balloon create an isodiametric fit with the stomach wall surrounding the incision, facilitating advancement of the cannula through the incision. In an alternative embodiment shown in FIG. 18, the proximal portion of the balloon may include a proximal taper 222 to facilitate advancement of the cannula by orienting the edges of the incision towards the cannula 10. FIG. 19 illustrates that the dilation balloon 220 may include an outer annular balloon 224 that expands in a proximal direction, driving tissue surrounding the incision over the edges of the cannula 10. Once the incision I has been dilated, the cannula 10 is advanced through the incision and the anchoring balloons 20a, 20b are expanded as discussed above. FIG. 17K.

In a slight modification to the method described in connection with FIGS. 17 A-17I, the obturator and septum may be retracted prior to penetration using the needle 218 so as to create suction against the stomach wall, thus provided counter-traction for the advancement of the needle. In either case, suction may be applied through the obturator or access cannula to engage the stomach wall for penetration.

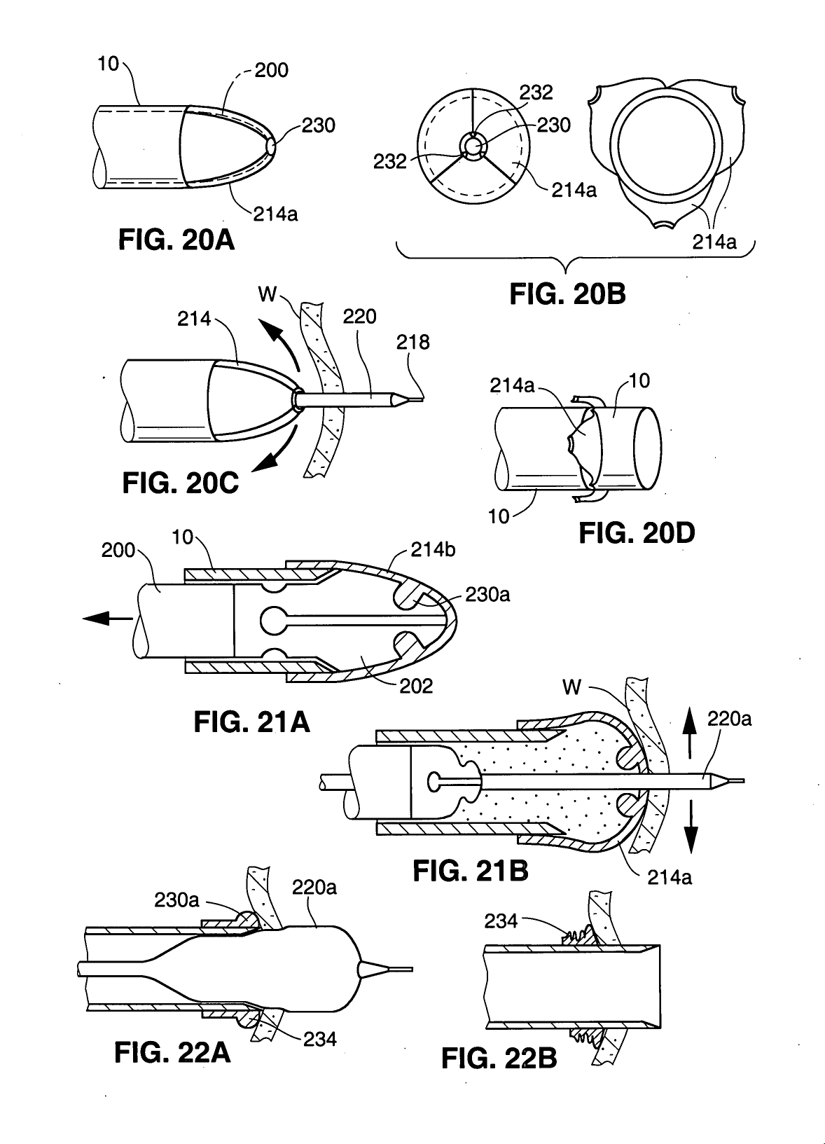

FIG. 20A shows an alternative access system for use in aseptically positioning the access cannula 10. The FIG. 20A system, which is similar to the FIG. 16 system, includes cannula 10, obturator 200, a balloon dilator 220 having a retractable needle tip 218, and a septum 214a. In this embodiment, the obturator and septum are independent structures. The tip of the septum 214a includes an o-ring 230 having notches 232. The center of the o-ring is covered by the septum to seal the distal end of the cannula and obturator. During use of the FIG. 20A embodiment, needle 218 and balloon dilator 220 are advanced through the o-ring 230, penetrating the septum 214a and the stomach wall W as shown in FIG. 20C. Expansion of balloon dilator 220 ruptures the o-ring 230 and the septum as shown in FIG. 20D.

Another alternative embodiment shown in FIGS. 21A through 22B is similar to the FIG. 20A embodiment in that the balloon dilator 220 is used to rupture the septum 214b. Referring to FIG. 21B, after the obturator 202 is retracted, the septum 214b is pressurized and stretched to a tensioned state using sterile saline. When the septum 214b is penetrated and ruptured using the balloon dilator, the ruptured septum gathers on the exterior of the cannula 10, forming a stop 234 to prevent inadvertent advancement of the cannula 10 further into the stomach, and additionally forming a seal around the incision. O-ring 230a may be sufficiently large that it will not rupture in response to expansion of the dilator, but will instead retract towards the exterior surface of the cannula when the septum is ruptured.

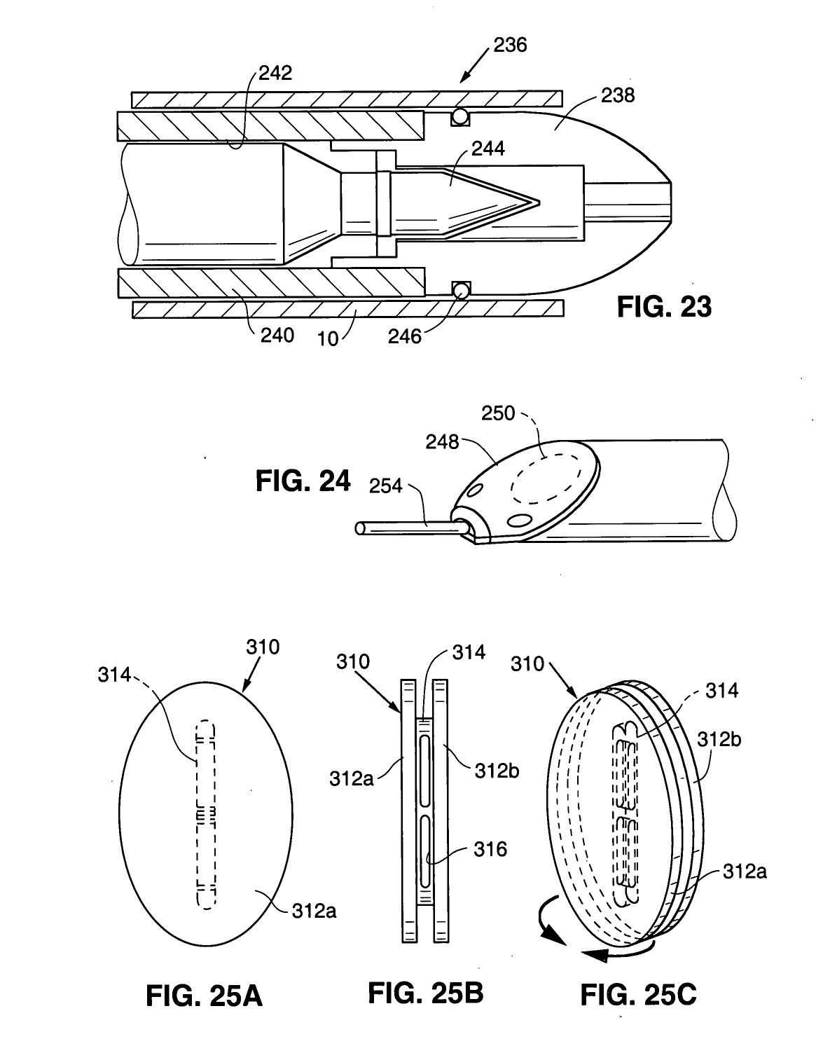

As illustrated in FIG. 23, an alternative obturator 236 includes a tapered tip 238 on a braided shaft 240. A lumen 242 in the shaft 240 and tip 238 is fluidly coupled to a duckbill valve 244, which remains closed except when the needle and balloon dilator are passed through it. An o-ring seal 246 seals the obturator against the interior surface of the cannula 10.

FIG. 24 illustrates a dilator that may be used with any of the disclosed embodiments. Dilator 248 includes a tip having an off-set taper. A transparent window 250 is positioned to allow viewing of the target tissue using an endoscope although the entire dilator tip may also be transparent. Flush ports 252 are positioned to direct a sterile saline solution or an antiseptic agent into contact with the stomach wall before and/or during penetration of the wall. A needle sheath 254 having a safety needle extendable from it is used to penetrate the stomach wall.

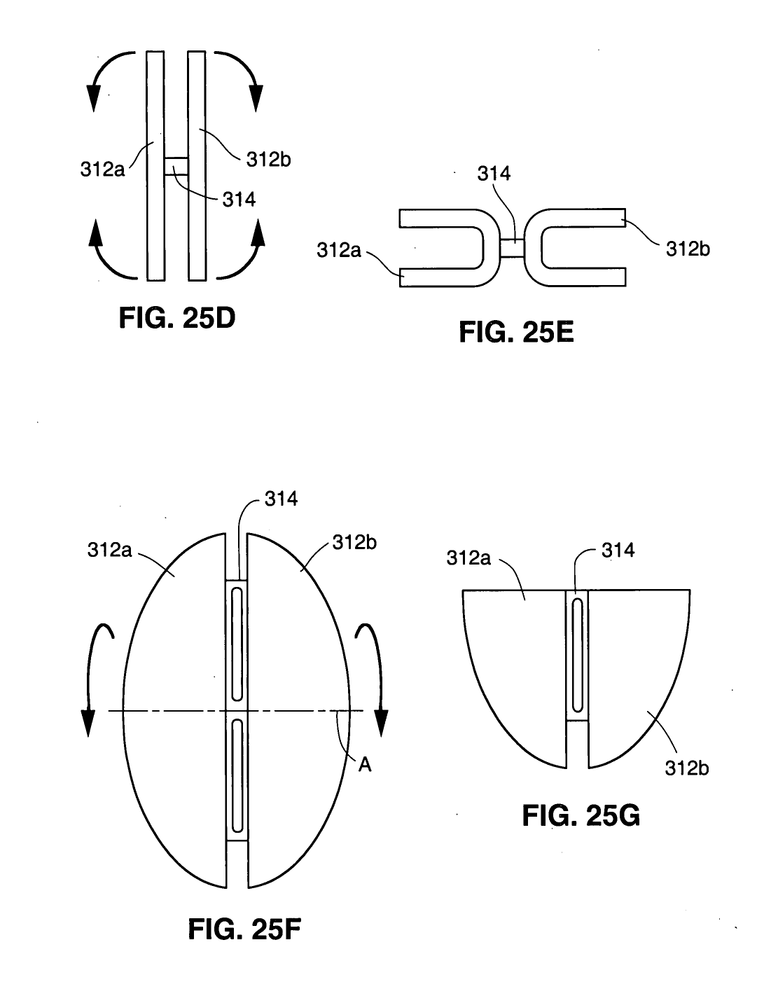

As discussed earlier, the anchors described above may be left behind to close the incision formed in the stomach wall or the wall of another body cavity. FIGS. 25A-25C show other closure devices that may be endoscopically implanted to close the incision formed in the stomach wall or other body wall. For simplicity, any type of opening formed in the body wall (including but not limited to the dilated needle punctures described above) will be referred to as an incision. In general, the closure devices comprise a pair of expandable portions, one of which is positioned inside the stomach and the other of which is positioned on the stomach exterior. A connecting feature extends between the expandable portions and is generally positioned extending through the incision. The closure devices seal the incision preventing passage of fluids or material from stomach into the peritoneal cavity. They are preferably bioabsorbable/bioerodible implants, but may instead be permanent implants.

FIGS. 25A-25C illustrate one exemplary embodiment of a closure device 310, which includes a pair of wings 312a, 312b and a connecting element 314 of any of a number of shapes extending between the wings. Wings 312a, 312b are shown as having an oval shape, although other shapes including, but not limited to, elliptical or circular shapes may be used. In the first embodiment, the connecting element 314 is an elongate rib proportioned so that it may be positioned within an incision in the stomach. While not mandatory, the elongate shape of the rib is particularly suitable for a closure device used to close an elongate cut or tear in the tissue. The dimensions for the closure device are selected such that the spacing between the wings is sufficient to seal the incision without imparting excessive compressive forces on the stomach wall tissue. In one embodiment, the separation between the opposed surfaces of the wings is in the range of 0.06-0.1 inches.

The materials for the wings and rib are preferably materials that will bioerode, degrade or absorb after a period of time calculated to allow healing of the incision. Preferred materials include but are not limited to bioerodible elastomers or biorubbers such as those formed using sebacic acid materials. Mesh, braid or woven materials formed using absorbable suture material may also be used. If mesh, braid or woven components are used for sealing components (e.g. one or both of the wings), they are desirably of sufficiently tight construction to prevent fluid passage through them, or they are sealed against fluid passage using bioabsorbable adhesives or other structures. The closure devices may be constructed with various combinations of materials. As one example, a device may have bioabsorbable polymer wings and a bioabsorbable mesh connector element. Additionally, each feature may have combinations of materials—such as a biopolymer reinforced by an embedded absorbable mesh structure. The materials may be coated or impregnated using sclerosing agents or other materials that will promote healing of the stomach wall tissue.

Ribs 314 may be provided with pores, openings or other features through which tissue may grow as the stomach tissue heals. In the FIG. 25A-25C embodiment, such features are in the form of slots 316.

The closure device 310 is constructed so it may be folded for insertion into a tube for deployment. Various folding arrangements may be used. One example is shown in FIGS. 25D-25F. FIG. 25D is a top view of the closure device prior to folding. As indicated by arrows, each wing 312a, 312b is first folded onto itself along its longitudinal axis, configuring the device 10 as shown in the top view of FIG. 25E and the side view of FIG. 25F. Next, with reference to FIG. 25F, the upper portion of the device 310 is folded across the horizontal axis A so that each wing 312a, 312b is again folded over on itself, placing the device 310 into the configuration shown in FIG. 25G.

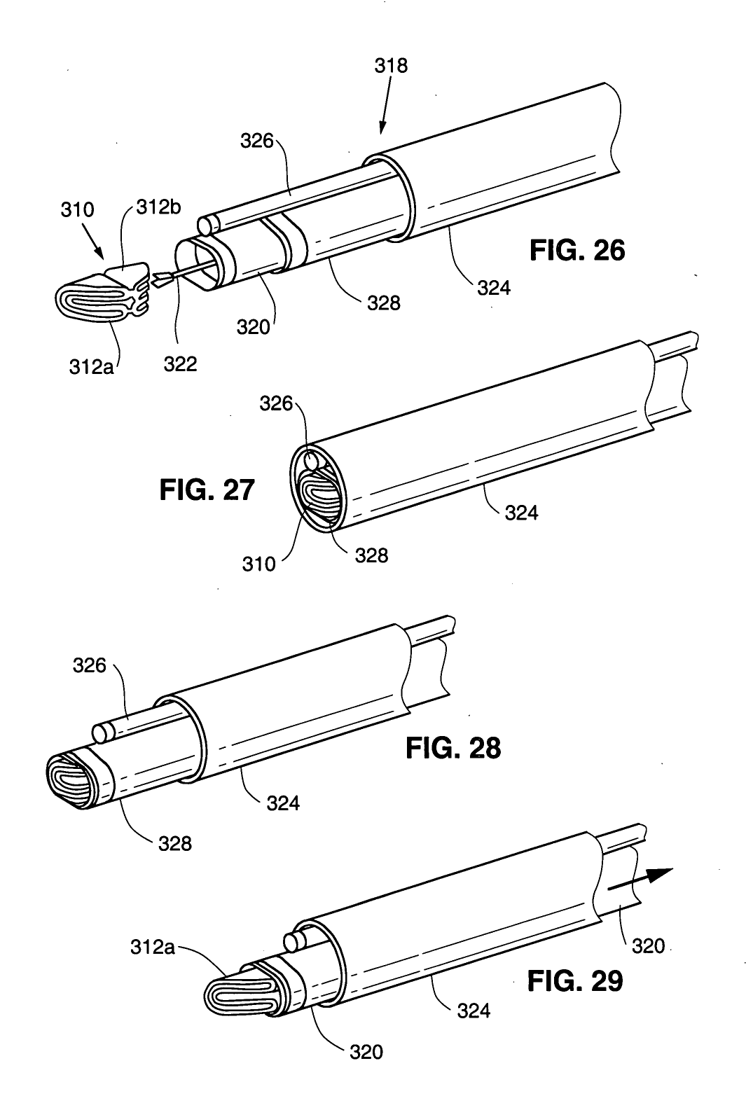

FIG. 26 illustrates a deployment system 318 of a type that may be used for implanting the closure device 310. System 318 includes a delivery cannula 320, a grasper 322 extending through cannula 320, a outer sheath 324, an endoscope 326 and an intermediate sheath 328. Use of the system 318 will next be described.

In preparation for deployment, the closure device 310 is folded as described above, and the wing 312b to be deployed in the stomach interior is engaged in its folded state by grasper 322. The grasper 322 and a portion of the device 310 (including wing 312b) is withdrawn into the delivery cannula 320, leaving wing 312a positioned outside the distal opening of the delivery cannula 320. The delivery cannula 320 and the folded closure device 310 are positioned within the intermediate sheath 328 so as to maintain the folded configuration of the device 310. The intermediate sheath 328 and endoscope are positioned within the outer sheath 324 as shown in FIG. 27.

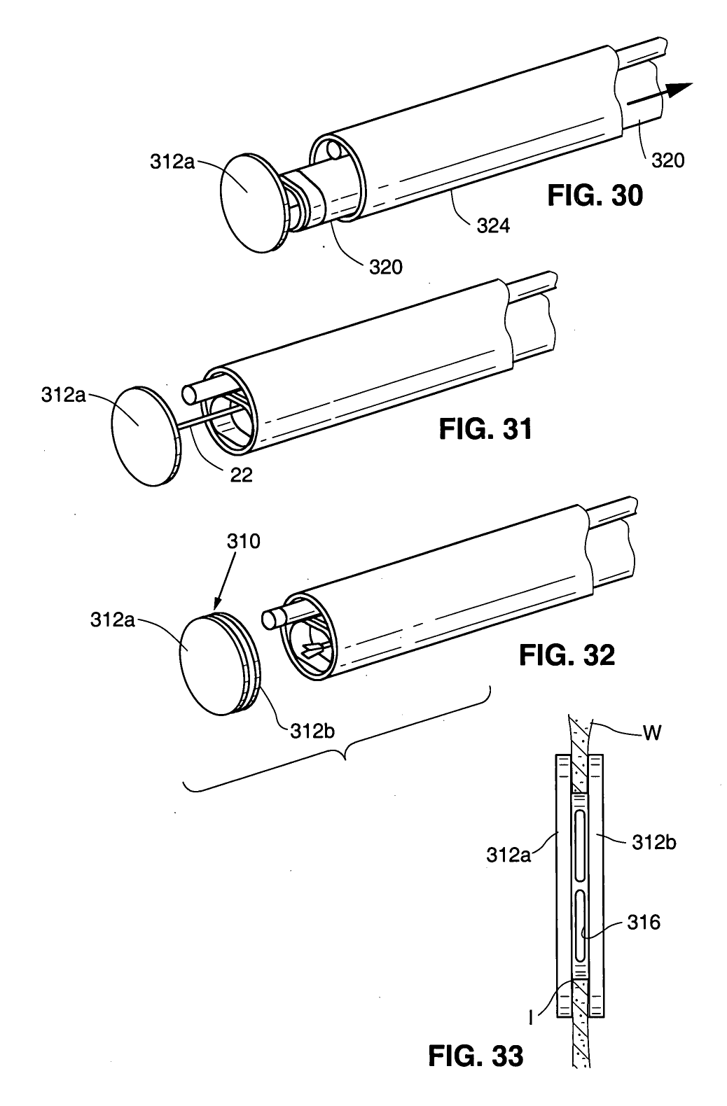

The distal end of the outer sheath 324 is passed through the mouth and esophagus and into the stomach. As shown in FIG. 28, the intermediate sheath 328 is advanced out of the outer sheath 324 and through the incision (not shown) under visualization using the endoscope 326. At this stage the device 310 is within the intermediate sheath 328, along with the grasper 322 and delivery cannula 320, neither of which is visible in FIG. 28. Referring to FIG. 29, the intermediate sheath 328 is next withdrawn, exposing the wing 312a of the device 310, causing the wing to expand on the exterior of the stomach to the position shown in FIG. 29. The delivery cannula 320 is withdrawn as shown in FIG. 30, but the wing 312b remains folded because it remains within the jaws of the grasper 322. Traction is applied to the grasper to pull the external wing 312a into contact with the stomach wall. The grasper 322 is then actuated to release the wing 312b, causing it to expand in the stomach interior (FIG. 32), leaving the device positioned within the incision as shown in FIG. 33. One or both of the wings 312a, 312b forms a seal with the stomach wall to prevent leakage of stomach contents into the peritoneal space. As the incision heals, tissue grows through the slots 316. Over time, the device degrades or absorbs within the body.

In the system for deploying the closure devices, the delivery cannula 320 may be the access cannula 10 of FIG. 1 or a separate cannula. If the closure device is deployed while the access cannula 10 is in place, the anchoring elements 20a, 20b will be deflated at appropriate times to make way for the wings of the closure device.

FIGS. 34 and 35 shown an alternative embodiment of an access cannula 400, which includes an inner cannula section that remains in a sterile environment until it is passed through the deployed anchors 20a, 20b and into the peritoneal cavity. Specifically, cannula 400 includes a tubular proximal section 402 having a lumen 404, and a distal section 406 that is longitudinally compressible from the elongated position shown in FIG. 34 to the compressed position shown in FIG. 35. An inner cannula 408 extends longitudinally from the proximal section 402 and includes a lumen 410 in communication with lumen 404 of the proximal section 404. When the cannula distal section 406 is in the elongated position, the inner cannula 408 is fully within the distal section 406, allowing sterility of the inner cannula 408 during movement of the cannula 400 through the mouth, esophagus and stomach. After the anchors 20a, 20b are deployed as described above, the distal section 406 is compressed by axially loading the cannula 400 in the direction of the arrow A in FIG. 35. Compression of the distal section causes inner cannula 408 to exit the distal section 406 (via valve 418 if one is provided as in FIG. 4) and to protrude into the peritoneal cavity, allowing sterile access to the peritoneal cavity via lumens 404 and 410.

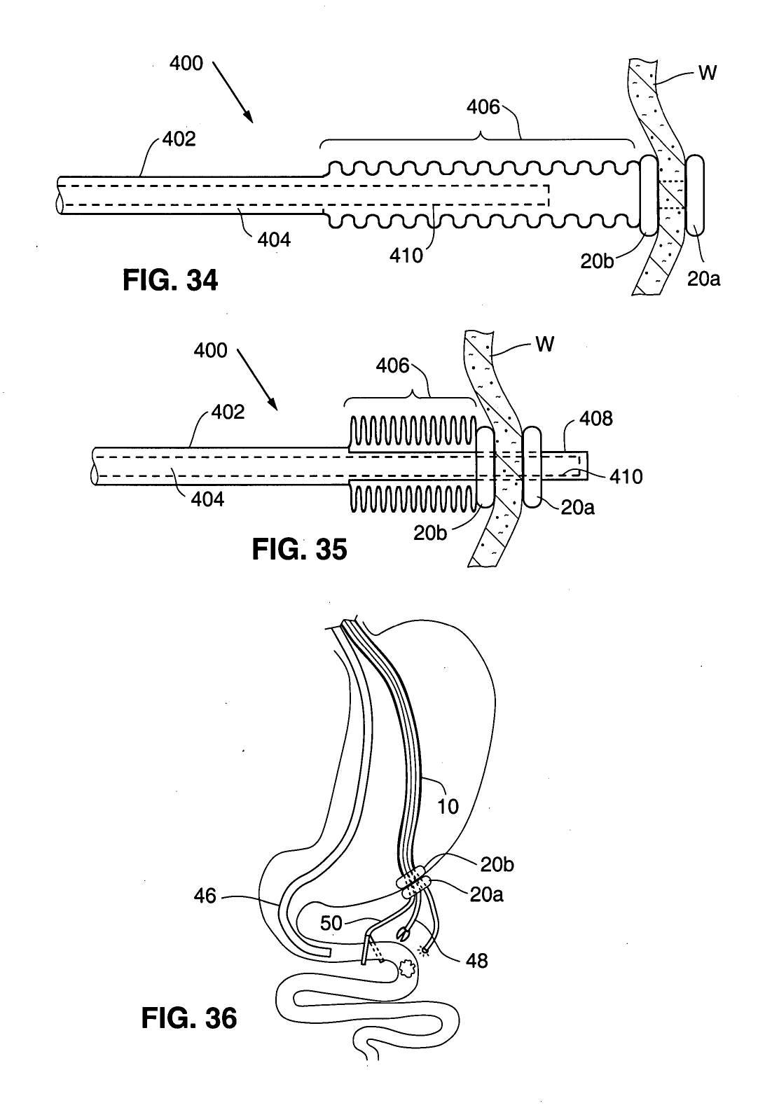

Referring to FIG. 36, the access cannula 10 (or cannula 100) may be used for introduction of instruments used to perform surgery on the bowel B, such as bowel resection to remove a diseased portion of the bowel. As shown, an intraluminal endoscope 46 is passed transorally into the stomach and into the intestine, allowing the surgeon to identify diseased or injured sections of the bowel. A grasper 48 passed into the peritoneal cavity via access cannula 10 may be used to manipulate the bowel into a desired position for treatment, and/or it may be used to pull a target region of the bowel over the intraluminal endoscope 46 for inspection. An endoscopic stapler 50 introduced through the access cannula 10 can be used to resect and/or staple a portion of the bowel, and a camera 52 may be used for visualization of the procedure. Instruments (e.g. staplers, endoscopes, and/or others) may also be introduced through one or more laparoscopic ports providing access to the surgical cavity.

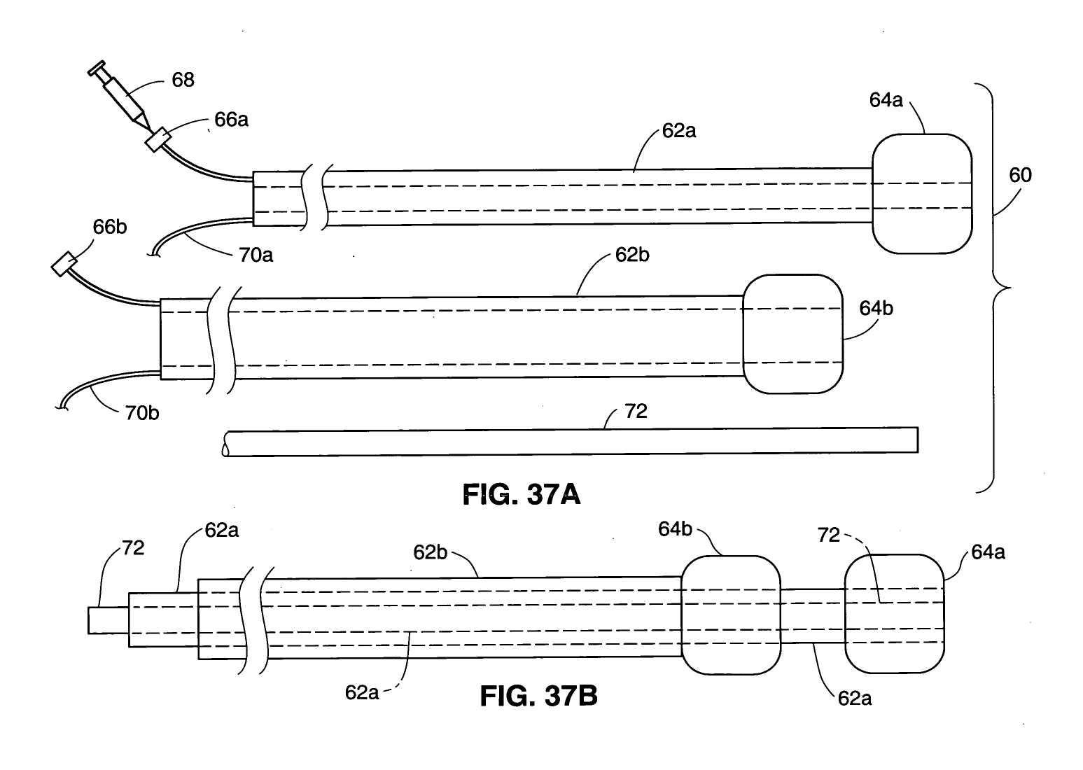

As discussed in connection with FIG. 36, if it is desired to inspect the bowel using a transorally introduced endoscope, manipulation of the bowel may be necessary in order to bring portions of the bowel into the viewing range of the endoscope. FIG. 37A illustrates a system 60 that allows for such manipulation and inspection from within the bowel. As shown, system 60 includes a pair of flexible elongate tubular members 62a, 62b, each of which includes an inflatable balloon 64a, 64b on its distal end. Balloons 64a, 64b are constructed of a size and material that will allow them to engage the interior wall of the intestine when they are inflated from inside the intestine. The exterior surfaces of the balloons 64a, 64b may include surface features (for example, textures, ridges, barbs, or fish scale type structures) that facilitate engagement of the intestinal wall.

Inflation ports 66a, 66b are provided for inflating the balloons using a syringe 68 or other inflation device. Guide wires 70a, 70b may also extend through lumens in the tubular members. As shown in FIG. 37B, the tubular members 62a, 62b and endoscope 72 are arranged such that the endoscope 72 extends through the lumen of the tubular member 62b, and the tubular member 62b extends through the lumen of the tubular member 62a. The system may include one or more elements (not shown) for locking the positions of the tubular members 62a, 62b (and/or the endoscope 72) relative to one another.

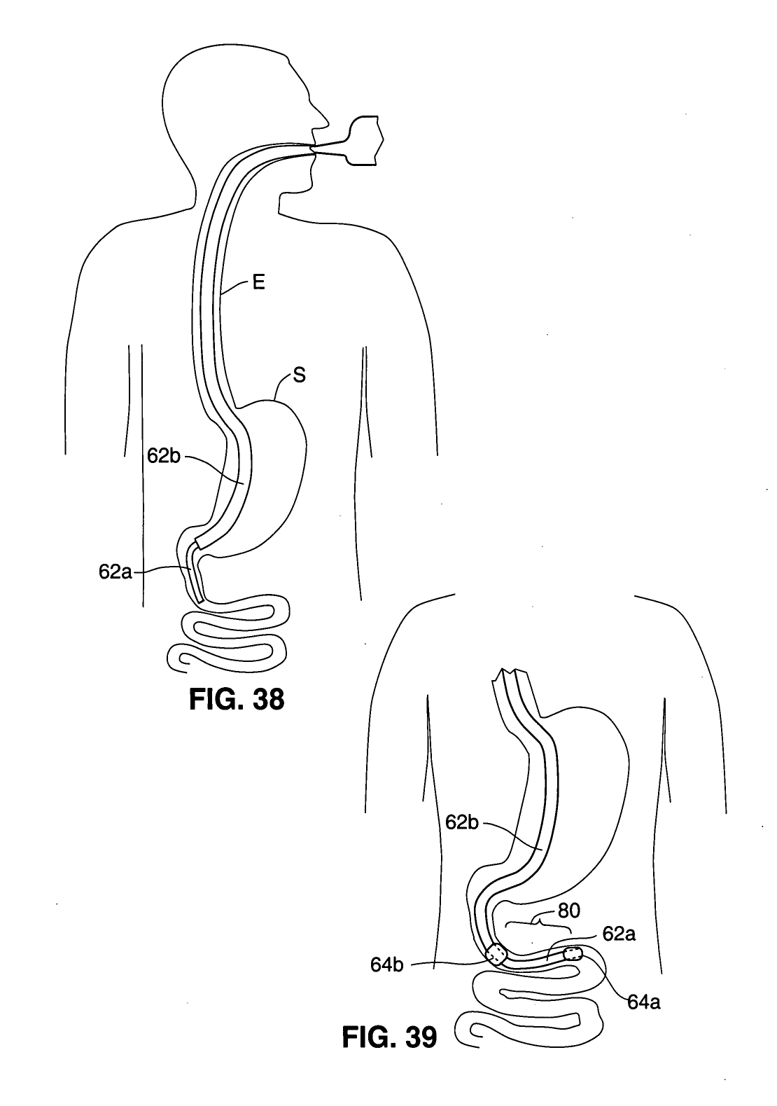

FIGS. 38 through 42 illustrate use of the bowel manipulation device of FIG. 37A. First, the components are arranged as shown in FIG. 37B, but with the balloons 64a, 64b in their deflated state. The assembled components are introduced into the intestine via the esophagus and stomach. Once the system is within the intestine, balloon 64b is inflated as shown in FIG. 39. However, before the tubular member 62a is advanced to the position shown in FIG. 39, endoscope 72 is advanced out of the tubular members and used to inspect the section of intestine 80.

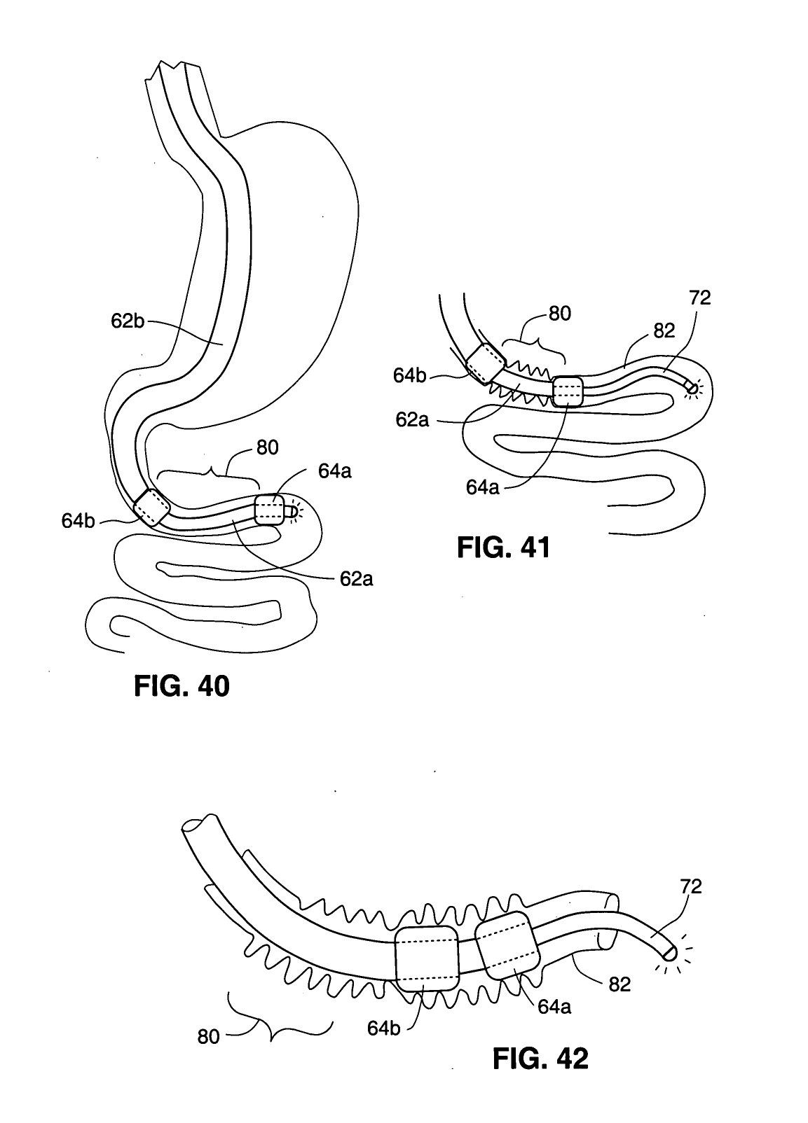

Next, tubular member 62a is advanced further to a more distal region of the intestine (FIG. 39), and then balloon 64a is inflated as shown in FIG. 40. With both balloons inflated, tubular member 62a is retracted in a proximal direction as indicated by an arrow in FIG. 40, causing balloon 64a to carry a section of the intestine in a proximal direction, thereby compressing the previously inspected section of bowel 80 and thus causing a distally adjacent section of bowel 82 to be presented within the viewing range of scope 72. See FIG. 41. Once section 82 is inspected, balloon 64b is deflated and tubular member 62b is advanced to move balloon 64b into position adjacent to balloon 64a as shown in FIG. 42. Repositioned balloon 64b retains the previously retracted bowel section 82 in its retracted state, thus allowing repositioning of balloon 64a without releasing retracted section 80. The scope 72 is advanced distally to a new position, and then balloon 64a is then deflated, advanced distally, reinflated and then retracted towards balloon 64b, thus retracting bowel section 82 while presenting another section of the intestine within view of the scope 72. The method is repeated as required to permit viewing of as much of the intestine as needed.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Moreover, various features of the disclosed embodiments may be combined with one other or with additional features to create additional embodiments falling within the scope of the present invention.

Any and all patents, patent applications and printed publications referred to above, including those relied upon for purposes of priority, are incorporated by reference.

We claim:

1. A method of performing surgery within a living body, comprising the steps of:
inserting an access cannula through a natural orifice into a hollow body organ, the access cannula having a distal end;
using an incising instrument extending through the access cannula, forming an incision in a wall of the hollow body organ;
anchoring a distal portion of the access cannula within the incision such that the distal end is within a body cavity outside the hollow body organ;
inserting surgical instruments through the access cannula into the body cavity and carrying out a procedure within the body cavity; and
withdrawing the access cannula from the hollow body cavity and out the natural orifice, leaving a closure device within the incision.

2. The method of claim 1, wherein the closure device is a bioerodible or biodegradable closure device.

3. The method of claim 1, wherein the method further includes positioning an obturator within the access cannula, and wherein the inserting step includes inserting the access cannula and obturator through the natural orifice and into the hollow body organ.

4. The method of claim 3, wherein the forming step includes forming the incision using an incising instrument passed through the obturator.

5. The method of claim 4, wherein the forming step further includes the step of expanding a dilator within the incision.

6. The method of claim 1, wherein the anchoring step includes passing a distal portion of the access cannula through the incision into the body cavity and expanding an anchor on the distal portion.

7. The method of claim 6, wherein the expanding step includes expanding an anchoring balloon.

8. The method of claim 6, wherein the expanding step includes expanding an anchoring braid.

9. The method of claim 6, wherein the expanding step includes expanding an anchor comprising a disk element.

10. The method of claim 6, wherein the leaving step includes the step of detaching the anchor from the cannula, wherein the anchor comprises the closure device.

11. An access system for natural orifice surgery, comprising:
an access cannula having a distal opening;
an obturator having a tapered distal tip, the obturator positionable within the access cannula with the distal tip extending from the distal opening; and
a seal sealing the distal opening of the access cannula.

12. The system of claim 11, wherein the seal is a septum covering the distal opening.

13. The system of claim 12, further including an incising element advanceable out the distal opening of the access cannula through the septum and a body wall positioned adjacent to the distal opening to form an incision in the body wall.

14. The system of claim 13, further including a dilator advanceable out the distal opening of the access cannula into an incision in the body wall, the dilator expandable to dilate the incision.

15. The system of claim 12, wherein the septum is coupled to the obturator, and wherein the obturator is retractable within the access cannula to retract the septum.

16. The system of claim 11, wherein the seal is a sealing ring contacting an exterior surface of the obturator and an interior surface of the access cannula.

17. The system of claim 11, further including an incising element advanceable out the distal opening of the access cannula and through a body wall positioned adjacent to the distal opening.

18. The system of claim 17, further including a dilator advanceable out the distal opening of the access cannula into an incision in the body wall, the dilator expandable to dilate the incision.

19. The system of claim 11, wherein the access cannula is proportioned to extend from a mouth, through an esophagus to a stomach wall in a human patient.

20. The system of claim 11, wherein the seal is a one-way valve.

21. The system of claim 11, further including at least one pull wire extending through the wall of the access cannula, the pull wire coupled to a distal portion of the access cannula for deflection thereof.

22. The system of claim 11, wherein the access cannula is formed of compliant material.

23. The system of claim 11, wherein the access cannula is formed of a porous material.

24. The system of claim 23, further including a source of sterile agent injectable through the access cannula, the agent passable through pores in the porous material.

25. The system of claim 11, wherein access cannula is formed of ePTFE

26. An access cannula for natural orifice surgery, comprising:
an elongate tubular member having a proximal section having a lumen and a distal section including a distal opening, wherein the distal section includes an inner cannula fluidly coupled to the lumen and an elongate bellows section disposed around the inner cannula, the bellows section compressible to cause the inner cannula to extend from the distal opening, and expandable to retract the inner cannula into the elongate bellows.

27. The access cannula according to claim 26, further including an expandable anchor on the exterior of the distal section, the expandable anchor expandable into contact with an internal body wall surrounding an incision.

28. The access cannula according to claim 26, further including a seal sealing the distal opening, wherein compressing the bellows section causes the inner cannula to advance through the seal.

29. The access cannula according to claim 20, wherein the inner cannula has a sharpened distal tip sufficient to penetrate a body wall when extended into contact with a body wall.

30. The access cannula according to claim 29, further including a seal sealing the distal opening, wherein compressing the bellows section causes the inner cannula to advance through the seal and through a body wall adjacent the distal opening.

31. A method of gaining natural orifice access to a body cavity, comprising the steps of:
providing an access cannula having a lumen and a distal opening, and an obturator positionable within the lumen;
with the obturator within the lumen, inserting the access cannula and obturator through the natural orifice and into the hollow body organ;
using an incising instrument extending through the access cannula, forming an incision in a wall of the hollow body organ; and
anchoring a distal portion of the access cannula within the incision such that the distal end is within a body cavity outside the hollow body organ.

32. The method of claim 31, wherein the providing step provides a seal sealing the distal opening.

33. The method of claim 32, wherein the providing step provides the seal to be a septum covering the distal opening, and wherein the step of forming the incision advances the incising instrument through the septum.

34. The method of claim 33, wherein the step of advancing the incising instrument through the septum ruptures the septum.

35. The method of claim 31, wherein the providing step provides the seal to be an annular seal positioned between the access cannula and the obturator.

36. The method of claim 31, further including the step of retracting the obturator from the distal opening.
